(19) United States
(12) Patent Application Publication
(10) Pub. No.: US 2004/0138305 A1
(43) Pub. Date: Jul. 15, 2004

(54) Alpha2delta ligands for the treatment of fibromyalgia and other disorders

(76) Inventors: Charles Price Taylor JR., Chelsea, MI (US); Andrew John Thorpe, Whitmore Lake, MI (US); Fong Wang, Ann Arbor, MI (US); David Juergen Wustrow, Ann Arbor, MI (US)

Correspondence Address: WARNER-LAMBERT COMPANY, 2800 PLYMOUTH RD, ANN ARBOR, MI, 48105, US

(21) Appl. No.: 10/731,225

(22) Filed: Dec. 9, 2003

Related U.S. Application Data
(60) Provisional application No. 60/433,491, filed on Dec. 13, 2002.
(60) Provisional application No. 60/483,482, filed on Jun. 27, 2003.

(51) Int. Cl. A61K 31/195

(52) U.S. Cl. 514/561; 514/567

(57) ABSTRACT

The invention relates to a method of treating fibromyalgia and other disorders in a mammal by administering a compound compound of Formula I

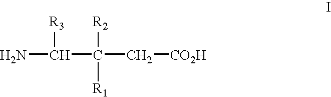

R₁ is straight or branched unsubstituted alkyl of from 1 to 5 carbon atoms, unsubstituted phenyl, or unsubstituted cycloalkyl of from 3 to 6 carbon atoms;

R₂ is hydrogen or methyl; and

R₃ is hydrogen, methyl, or carboxyl, or a pharmaceutically acceptable salt thereof.

This application claims priority from U.S. provisional application Serial No. 60/4833,491 filed Dec. 13, 2002 and U.S. provisional application Serial No. 60/483,482 filed Jun. 27, 2003; the entire contents of which are hereby incorporated herein by reference.

This invention relates to methods of treating various central nervous system and other disorders by administering certain compounds that exhibit activity as calcium channel alpha2delta ligands (“α2δ ligands” or “alpha2delta ligands”). Such compounds have affinity for the α2δ subunit of a calcium channel. Such compounds have also been referred to in the literature as gamma-aminobutyric acid (GABA) analogs.

### BACKGROUND OF THE INVENTION

Several alpha2delta ligands are known. Gabapentin, a cyclic alpha2delta ligand, is now commercially available (Neurontin®, Warner-Lambert Company) and extensively used clinically for treatment of epilepsy and neuropathic pain. Such cyclic alpha2delta ligands are described in U.S. Pat. No. 4,024,175, which issued on May 17, 1977, and U.S. Pat. No. 4,087,544, which issued on May 2, 1978. Other series of alpha2delta ligands are described in U.S. Pat. No. 5,563,175, which issued on Oct. 8, 1996, U.S. Pat. No. 6,316,638, which issued on Nov. 13, 2001, U.S. Provisional Patent Application 60/353,632, which was filed on Jan. 31, 2002, U.S. Provisional Patent Application 60/248,630, which was filed on Nov. 2, 2002, U.S. Provisional Patent Application 60/421,868, which was filed on Oct. 28, 2002, U.S. Provisional Patent Application 60/421,867, which was filed on Oct. 28, 2002, U.S. Provisional Patent Application 60/413,856, which was filed on Sep. 25, 2002, U.S. Provisional Patent Application 60/411,493, which was filed on Sep. 16, 2002, US Provisional Patent Application 60/421,866, which was filed on Oct. 28, 2002, U.S. Provisional Patent Application 60/441,825, which was filed on Jan. 22, 2003, U.S. Provisional Patent Application 60/452,871, which was filed on Mar. 7, 2003, European Patent Application EP 1112253, which was published on Jul. 4, 2001, PCT Patent Application WO 99/08671, which was published on Feb. 25, 1999, and PCT Patent Application WO 99/61424, which was published on Dec. 2, 1999. These patents and applications are incorporated herein by reference in their entireties.

Additional uses for alpha2delta ligands, including compounds of the formula I, which are defined below, are referred to in U.S. Provisional Patent Application 60/433,491, which was filed on Dec. 13, 2002. This application is incorporated herein by reference in its entirety.

### SUMMARY OF THE INVENTION

This invention relates to a method of treating fibromyalgia in a mammal, preferably a human, comprising administering to a mammal in need of such treatment a therapeutically effective amount of an alpha2delta ligand of the formula I

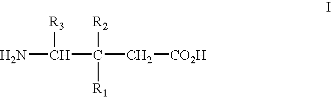

or a pharmaceutically acceptable salt thereof, wherein:

R₁ is a straight or branched unsubstituted alkyl of from 1 to 5 carbon atoms, unsubstituted phenyl, or unsubstituted cycloalkyl of from 3 to 6 carbon atoms;

R₂ is hydrogen or methyl; and

R₃ is hydrogen, methyl, or carboxyl.

Fibromyalgia (FM) is a chronic syndrome characterized mainly by widespread pain, unrefreshing sleep, disturbed mood, and fatigue. Other syndromes commonly comorbid with fibromyalgia include irritable bowel syndrome, migraine headaches, depression and insomnia, among others. Success of treating fibromyalgia with a single pharmacological agent has been characterized as modest and results of clinical trials have been characterized as disappointing. It is believed that based on current understanding of the mechanisms and pathways involved in fibromyalgia, multiple agents will be required, aimed at the major symptoms of pain, disturbed sleep, mood disturbances, and fatigue. Fibromyalgia patients are often sensitive to side effects of medications, a characteristic perhaps related to the pathophysiology of this disorder (Barkhuizen A, Rational and Targeted pharmacologic treatment of fibromyalgia. Rheum Dis Clin N Am 2002; 28: 261-290; Leventhal L J. Management of fibromyalgia. Ann Intern Med 1999;131:850-8).

While fibromyalgia is a complex disorder with multiple facets, this complexity can be well assessed (Yunus M B, A comprehensive medical evaluation of patients with fibromyalgia syndrome, Rheum Dis N Am 2002; 28:201-217). The diagnosis of FM is usually based on the 1990 recommendations of the American College of Rheumatology classification criteria (Bennett R M, The rational management of fibromyalgia patients. Rheum Dis Clin N Am 2002; 28: 181-199; Wolfe F, Smythe H A, Yunus M B, Bennett R M, Bombardier C, Goldenberg D L, et al. The American College of Rheumatology 1990 criteria for the classification of fibromyalgia: Report of the Multicenter Criteria Committee. Arthritis Rheum 1990; 33:160-72). Evaluation, management, and pharmacological treatment of fibromyalgia have been reviewed (Barkhuizen A, Rational and Targeted pharmacologic treatment of fibromyalgia. Rheum Dis Clin N Am 2002; Buskila D, Fibomyalgia, chronic fatigue syndrome and myofacial pain syndrome. Current opinions in Rheumatology 2001; 13: 117-127; Leventhal L J. Management of fibromyalgia. Ann Intern Med 1999;131:850-8; Bennett R M, The rational management of fibromyalgia patients. Rheum Dis Clin N Am 2002; 28: 181-199; Yunus M B, A comprehensive medical evaluation of patients with fibromyalgia syndrome, Rheum Dis N Am 2002; 28:201-217).

A more specific method of this invention relates to the above method of treating fibromyalgia wherein a compound of the formula I, or a pharmaceutically acceptable salt thereof, is administered to a human for the treatment of fibromyalgia that is accompanied by one or more somatic symptoms selected from fatigue, headache, neck pain, back pain, limb pain, joint pain, abdominal pain, abdominal distention, gurgling, diarrhea nervousness, and the symptoms associated with generalized anxiety disorder (e.g., excessive anxiety and worry (apprehensive expectation), occurring more days than not for at least six months, about a number of events and activities, difficulty controlling the worry, etc.) See Diagnostic and Statistical manual of Mental Disorders, Fourth Edition (DSM-IV), American Psychiatric Association, Washington, D.C., May 1194, pp. 435-436 and 445-469.

This invention also relates to a method of treating a disorder or condition selected from the group consisting of sleep disorders such as insomnia (e.g., primary insomnia including psychophysiological and idiopathic insomnia, secondary insomnia including insomnia secondary to restless legs syndrome, Parkinson's disease or another chronic disorder, and transient insomnia), somnambulism, sleep deprivation, REM sleep disorders, sleep apnea, hypersomnia, parasomnias, sleep-wake cycle disorders, jet lag, narcolepsy, sleep disorders associated with shift work or irregular work schedules, deficient sleep quality due to a decrease in slow wave sleep caused by medications or other sources, and other sleep disorders in a mammal, comprising administering to a mammal in need of such treatment a therapeutically effective amount of a compound of the formula I, or a pharmaceutically acceptable salt thereof.

This invention also relates to a method of increasing slow wave sleep in a human subject comprising administering to a human subject in need of such treatment a therapeutically effective amount of a compound of the formula I or a pharmaceutically acceptable salt thereof.

This invention also relates to a method of increasing growth hormone secretion in a human subject comprising administering to a human subject in need of such treatment a therapeutically effective amount of a compound of the formula I or a pharmaceutically acceptable salt thereof.

This invention also relates to a method of increasing slow wave sleep in a human subject comprising administering to a human subject in need of such treatment:

(a) a compound of the formula I or a pharmaceutically acceptable salt thereof; and

(b) a human growth hormone or a human growth hormone secretagogue or a pharmaceutically acceptable salt thereof;

wherein the amounts of the active agents “a” and “b” are chosen so as to render the combination effective in increasing slow wave sleep.

A more specific embodiment of this invention relates to the above method wherein the human growth hormone secretagogue that is employed is 2-amino-N-[2-(3a-benzyl-2-methyl-3-oxo-2,3,3a,4,6,7-hexahydro-pyrazole[4,3-c]pyridin-5-yl)-1-benzyloxymethyl-2-oxo-ethyl]-2-methyl-proprionamide.

This invention also relates to a method of increasing slow wave sleep in a human subject being treated with an active pharmaceutical agent that decreases slow wave sleep, such as morphine or another opioid analgesic agent or a benzodiazepine, comprising administering to a human subject in need of such treatment:

(a) a compound of the formula I or a pharmaceutically acceptable salt thereof; and

(b) a human growth hormone or a human growth hormone secretagogue or a pharmaceutically acceptable salt thereof;

wherein the amounts of the active agents “a” and “b” are chosen so as to render the combination effective in increasing slow wave sleep.

A more specific embodiment of this invention relates to the above method wherein the human growth hormone secretagogue that is employed is 2-amino-N-[2-(3a-benzyl-2-methyl-3-oxo-2,3,3a,4,6,7-hexahydro-pyrazole[4,3-c]pyridin-5-yl)-1-benzyloxymethyl-2-oxo-ethyl]-2-methyl-proprionamide.

This invention also relates to a method of increasing slow wave sleep in a human subject being treated with an active pharmaceutical agent that decreases slow wave sleep, such as morphine or another opioid analgesic agent, comprising administering to such human subject an amount of a compound of the formula I, as defined above, or a pharmaceutically acceptable salt thereof, that is effective in increasing slow wave sleep.

This invention also relates to a method of treating irritable bowel syndrome in a mammal, preferably a human, comprising administering to a human subject in need of such treatment a therapeutically effective amount of a compound of the formula I, or a pharmaceutically acceptable salt thereof.

This invention also relates to a method of treating a disorder or condition selected from the group consisting of panic disorder with or without agoraphobia, agoraphobia without history of panic disorder, specific phobias (e.g., specific animal phobias), social anxiety disorder, social phobia, obsessive-compulsive disorder (OCD), and stress disorders including post-traumatic stress disorder and acute stress disorder in a mammal, comprising administering to a mammal in need of such treatment a therapeutically effective amount of a compound of the formula I, or a pharmaceutically acceptable salt thereof.

A more specific embodiment of this invention relates to the above method wherein the disorder or condition being treated is post-traumatic stress disorder.

Another more specific embodiment of this invention relates to the above method wherein the disorder or condition being treated is social phobia or social anxiety disorder.

Another more specific embodiment of this invention relates to the above method wherein the disorder or condition being treated is OCD.

It will be appreciated that for the treatment of panic disorder, phobias, OCD and stress disorders, the compounds of formula I may be used in conjunction with other antidepressant or anti-anxiety agents. Suitable classes of anti-depressant agent include norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists and a typical antidepressants. Suitable norepinephrine reuptake inhibitors include tertiary amine tricyclics and secondary amine tricyclics. Suitable examples of tertiary amine tricyclics include amitriptyline, clomipramine, doxepin, imipramine and trimipramine, and pharmaceutically acceptable salts thereof. Suitable examples of secondary amine tricyclics include amoxapine, desipramine, maprotiline, nortriptyline and protriptyline, and pharmaceutically acceptable salts thereof. Suitable selective serotonin reuptake inhibitors include fluoxetine, fluvoxamine, paroxetine and sertraline, and pharmaceutically acceptable salts thereof. Suitable monoamine oxidase inhibitors include isocarboxazid, phenelzine, tranylcypromine and selegiline, and pharmaceutically acceptable salts thereof. Suitable reversible inhibitors of monoamine oxidase include moclobemide, and pharmaceutically acceptable salts thereof. Suitable serotonin and noradrenaline reuptake inhibitors of use in the present invention include venlafaxine, and pharmaceutically acceptable salts thereof. Suitable CRF antagonists include those compounds described in International Patent Application Nos. WO 94/13643, WO 94/13644, WO 94/13661, WO 94/13676 and WO 94/13677. Suitable a typical anti-depressants include bupropion, lithium, nefazodone, trazodone and viloxazine, and pharmaceutically acceptable salts thereof. Suitable classes of anti-anxiety agents include benzodiazepines and 5-HT_{IA} agonists or antagonists, especially 5-HTIA partial agonists, and corticotropin releasing factor (CRF) antagonists. Suitable benzodiazepines include alprazolam, chlordiazepoxide, clonazepam, chlorazepate, diazepam, halazepam, lorazepam, oxazepam, and prazepam, and pharmaceutically acceptable salts thereof. Suitable 5-HT_{IA} receptor agonists or antagonists include, in particular, the 5-HT_{IA} receptor partial agonists buspirone, flesinoxan, gepirone and ipsapirone, and pharmaceutically acceptable salts thereof.

This invention also relates to a method of treating a disorder or condition selected from the group consisting of panic disorder with or without agoraphobia, agoraphobia without history of panic disorder, specific phobias (e.g., specific animal phobias), social anxiety disorder, social phobia, obsessive-compulsive disorder, and stress disorders including post-traumatic stress disorder and acute stress disorder in a mammal, preferably a human, comprising administering to a mammal in need of such treatment:

(a) a compound of the formula I, or a pharmaceutically acceptable salt thereof; and

(b) another compound that is an antidepressant or an antianxiety agent, or a pharmaceutically acceptable salt thereof;

wherein the amounts of the active agents “a” and “b” are chosen so as to render the combination therapeutically effective.

A more specific embodiment of this invention relates to any of the above methods wherein a therapeutic amount of a compound of the formula I, or a pharmaceutically acceptable salt thereof, is administered to a human for the treatment of any two or more comorbid disorders or conditions selected from those disorders and conditions the treatment of which is referred to in any of the above methods. This method is hereinafter also referred to as “the method for treating concomitant disorders”

Another more specific embodiment of this invention relates to the above method of treating concomitant disorders wherein a compound of the formula I, or a pharmaceutically acceptable salt thereof, is administered to a human for the treatment of fibromyalgia and concomitant panic disorder.

Another more specific embodiment of this invention relates to the above method of treating concomitant disorders wherein a compound of the formula I, or a pharmaceutically acceptable salt thereof, is administered to a human for the treatment of fibromyalgia and concomitant irritable bowel syndrome.

Another more specific embodiment of this invention relates to the above method of treating concomitant disorders wherein a compound of the formula I, or a pharmaceutically acceptable salt thereof, is administered to a human for the treatment of fibromyalgia and concomitant functional abdominal pain.

Another more specific embodiment of this invention relates to the above method of treating concomitant disorders wherein a compound of the formula I, or a pharmaceutically acceptable salt thereof, is administered to a human for the treatment of fibromyalgia and concomitant neuropathic pain.

Neuropathic pain is defined as pain initiated or caused by a primary lesion or dysfunction in the nervous system (International Association for the Study of Pain). Nerve damage can be caused by trauma and disease and thus the term ‘neuropathic pain’ encompasses many disorders with diverse aetiologies. These include but are not limited to, diabetic neuropathy, post herpetic neuralgia, back pain, cervical radiculopathy, cancer neuropathy, chemotherapy-induced neuropathy, HIV neuropathy, Phantom limb pain, Carpal Tunnel Syndrome, chronic alcoholism, hypothyroidism, trigeminal neuralgia, uremia, trauma-induced neuropathy, or vitamin deficiencies. Neuropathic pain is pathological as it has no protective role. It is often present well after the original cause has dissipated, commonly lasting for years, significantly decreasing a patients quality of life (Woolf and Mannion 1999 Lancet 353: 1959-1964). The symptoms of neuropathic pain are difficult to treat, as they are often heterogeneous even between patients with the same disease (Woolf & Decosterd 1999 Pain Supp. 6: S141-S147; Woolf and Mannion 1999 Lancet 353: 1959-1964). They include spontaneous pain, which can be continuous, or paroxysmal and abnormal evoked pain, such as hyperalgesia (increased sensitivity to a noxious stimulus) and allodynia (sensitivity to a normally innocuous stimulus).

Another more specific embodiment of this invention relates to the above method of treating concomitant disorders wherein a compound of the formula I, or a pharmaceutically acceptable salt thereof, is administered to a human for the treatment of fibromyalgia and concomitant premenstrual dysphoric disorder or premenstrual syndrome.

Another more specific embodiment of this invention relates to the above method of treating concomitant disorders wherein a compound of the formula I, or a pharmaceutically acceptable salt thereof, is administered to a human for the treatment of fibromyalgia and concomitant major depressive disorder.

Another more specific embodiment of this invention relates to the above method of treating concomitant disorders wherein a compound of the formula I, or a pharmaceutically acceptable salt thereof, is administered to a human for the treatment of fibromyalgia and concomitant dysthymia.

Another more specific embodiment of this invention relates to the above method of treating concomitant disorders wherein a compound of the formula I, or a pharmaceutically acceptable salt thereof, is administered to a human for the treatment of fibromyalgia and a concomitant somatoform disorder selected from somatization disorder, conversion disorder, body dysmorphic disorder, hypochondriasis, somatoform pain disorder, undifferentiated somatoform disorder and somatoform disorder not otherwise specified. See Diagnostic and Statistical manual of Mental Disortders, Fourth Edition (DSM-IV), American Psychiatric Association, Washington, D.C., May 1194, pp. 435-436.

Another more specific embodiment of this invention relates to the above method of treating fibromyalgia wherein a compound of the formula I, or a pharmaceutically acceptable salt thereof, is administered to a human for the treatment of fibromyalgia that is accompanied by one or more somatic symptoms selected from loss of appetite, sleep disturbances (e.g., insomnia, interrupted sleep, early morning awakening, tired awakening), loss of libido, restlessness, fatigue, constipation, dyspepsia, heart palpitations, aches and pains (e.g., headache, neck pain, back pain, limb pain, joint pain, abdominal pain), dizziness, nausea, heartburn, nervousness, tremors, burning and tingling sensations, morning stiffness, abdominal symptoms (e.g., abdominal pain, abdominal distention, gurgling, diarrhea), and the symptoms associated with major depressive disorder (e.g., sadness, tearfulness, loss of interest, ferafulness, helplessness, hopelessness, fatigue, low self esteem, obsessive ruminations, suicidal thoughts, impaired memory and concentration, loss of motivation, paralysis of will, reduced appetite, increased appetite).

The foregoing methods are also referred to herein, collectively, as the “inventive methods” or the “methods of this invention”.

Preferred embodiments of the invention methods utilize a compound of Formula I that is 3-aminomethyl-5-methyl-hexanoic acid or, especially, (S)-3-(aminomethyl)-5-methylhexanoic acid, which is known generically as pregabalin.

The term “alkyl”, as used herein, unless otherwise indicated, includes saturated monovalent hydrocarbon radicals having straight, branched or cyclic moieties or combinations thereof. Examples of “alkyl” groups include, but are not limited to, methyl, ethyl, propyl, isopropyl, butyl, iso- sec- and tert-butyl, pentyl, hexyl, heptyl, 3-ethylbutyl, cyclopropyl, cyclobutyl, cyclopentyl, cyclohexyl, cycloheptyl, norbornyl, and the like.

The term “cycloalkyl”, as used herein, refers to saturated monovalent carbocyclic groups containing from 3 to 8 carbons and are selected from cyclopropyl, cyclobutyl, cyclopentyl, cyclohexyl, cycloheptyl, and cyclooctyl, unless otherwise stated.

The term “treating”, as used herein, refers to reversing, alleviating, inhibiting the progress of, or preventing the disorder or condition to which such term applies, or preventing one or more symptoms of such condition or disorder. The term “treatment”, as used herein, refers to the act of treating, as “treating” is defined immediately above.

Compounds of the formula I may contain chiral centers and therefore may exist in different enantiomeric and diastereomeric forms. Individual isomers can be obtained by known methods, such as optical resolution, optically selective reaction, or chromatographic separation in the preparation of the final product or its intermediate. This invention relates to all optical isomers and all stereoisomers of compounds of the formula I, both as racemic mixtures and as individual enantiomers and diastereoismers of such compounds, and mixtures thereof, and to all pharmaceutical compositions and methods of treatment defined above that contain or employ them, respectively. Individual enantiomers of the compounds of formula I may have advantages, as compared with the racemic mixtures of these compounds, in the treatment of various disorders or conditions.

In so far as the compounds of formula I of this invention are basic compounds, they are capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The free base form of the compound may be regenerated by contacting the acid addition salt so formed with a base, and isolating the free base form of the compound in the conventional manner. The free base forms of compounds of the formula I prepared according to a process of the present invention differ from their respective acid addition salt forms somewhat in certain physical properties such as solubility, crystal structure, hygroscopicity, and the like, but otherwise such free base forms of the compounds and their respective acid addition salt forms are equivalent for purposes of the present invention.

Pharmaceutically acceptable acid addition salts of the basic compounds useful in the method of the present invention include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are salts of amino acids such as arginate and the like and gluconate, galacturonate (see, for example, Berge S. M. et al., “Pharmaceutical Salts,” J. of Pharma. Sci., 1977;66:1).

In so far as the compounds of formula I of this invention are acidic compounds, they are capable of forming a wide variety of different salts with various inorganic and organic bases. A base addition salt of an acidic compound useful in the method of the present invention may be prepared by contacting the free acid form of the compound with a sufficient amount of a desired base to produce the salt in the conventional manner. A pharmaceutically acceptable base addition salt of an acidic compound useful in the above inventive methods be prepared by contacting the free acid form of the compound with a nontoxic metal cation such as an alkali or alkaline earth metal cation, or an amine, especially an organic amine. Examples of suitable metal cations include sodium cation (Na⁺), potassium cation (K⁺), magnesium cation (Mg²⁺), calcium cation (Ca²⁺), and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, supra., 1977). The free acid forms of the compounds of formula I may be regenerated by contacting the base addition salt forms so formed with an acid, and isolating the free acid of the compound in the conventional manner. The free acid forms of the compounds useful in the above inventive methods differ from their respective salt forms somewhat in certain physical properties such as solubility, crystal structure, hygroscopicity, and the like, but otherwise they are equivalent to their respective free acids for purposes of the present invention.

Certain of the compounds useful in the methods of this invention can exist in unsolvated forms as well as solvated forms, including hydrated forms. In general, the solvated forms, including hydrated forms, are equivalent to unsolvated forms and are intended to be encompassed within the scope of the present invention.

Certain of the compounds useful in the methods of this invention can exist as two or more tautomeric forms. Tautomeric forms of the compounds may interchange, for example, via enolization/de-enolization and the like. A method of the present invention may utilize any tautomeric form of an alpha2delta ligand, or a pharmaceutically acceptable salt thereof, as well as mixtures thereof.

The present invention also includes the above inventive methods that employ isotopically labelled compounds that are identical to those recited in Formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances.

The effectiveness of an orally administered drug is dependent upon the drug's efficient transport across the mucosal epithelium and its stability in entero-hepatic circulation. Drugs that are effective after parenteral administration but less effective orally, or whose plasma half-life is considered too short, may be chemically modified into a prodrug form.

A prodrug is a drug that has been chemically modified and may be biologically inactive at its site of action, but which may be degraded or modified by one or more enzymatic or other in vivo processes to the parent bioactive form.

This chemically modified drug, or prodrug, should have a different pharmacokinetic profile than the parent drug, enabling easier absorption across the mucosal epithelium, better salt formulation and/or solubility, improved systemic stability (for an increase in plasma half-life, for example). These chemical modifications may be, for example:

1) ester or amide derivatives which may be cleaved by, for example, esterases or lipases. For ester derivatives, the ester is derived from the carboxylic acid moiety of the drug molecule by known means. For amide derivatives, the amide may be derived from the carboxylic acid moiety or the amine moiety of the drug molecule by known means;

2) peptides which may be recognized by specific or nonspecific proteinases (A peptide may be coupled to the drug molecule via amide bond formation with the amine or carboxylic acid moiety of the drug molecule by known means);

3) derivatives that accumulate at a site of action through membrane selection of a prodrug form or modified prodrug form; or

4) any combination of 1 to 3.

Current research in animal experiments has shown that the oral absorption of certain drugs may be increased by the preparation of “soft” quaternary salts. The quaternary salt is termed a “soft” quaternary salt since, unlike normal quaternary salts, e.g., R—N⁺(CH₃)₃, it can release the active drug upon hydrolysis.

“Soft” quaternary salts have useful physical properties compared with the basic drug or its salts. Water solubility may be increased compared with other salts, such as the hydrochloride, but more important there may be an increased absorption of the drug from the intestine. Increased absorption is probably due to the fact that the “soft” quaternary salt has surfactant properties and is capable of forming micelles and unionized ion pairs with bile acids, etc., which are able to penetrate the intestinal epithelium more effectively. The prodrug, after absorption, is rapidly hydrolyzed with release of the active parent drug.

The above inventive methods that employ prodrugs of compounds of formula I are included within the scope of this invention. Prodrugs and soft drugs are known in the art (Palomino E., Drugs of the Future, 1990;15(4):361-368). The last two citations are hereby incorporated by reference.

### DETAILED DESCRIPTION OF THE INVENTION

Alpha2delta ligands having the formula I, and the synthesis of such compounds, are described in U.S. Pat. No. 5,563,175 and U.S. Pat. No. 6,197,819, which are incorporated herein by reference in their entireties.

All that is required to practice the methods of this invention is to administer a compound of the formula I, or a pharmaceutically acceptable salt thereof, in an amount that is therapeutically effective to treat one or more of the disorders or conditions referred to above. Such therapeutically effective amount will generally be from about 1 to about 300 mg/kg body weight of the patient being treated. Typical doses will be from about 10 to about 5000 mg/day for an adult patient of normal weight. In a clinical setting, regulatory agencies such as, for example, the Food and Drug Administration (“FDA”) in the U.S. may require a particular therapeutically effective amount.

In determining what constitutes an effective amount or a therapeutically effective amount of a compound of the formula I, or a pharmaceutically acceptable salt thereof, for treating one or more of the disorders or conditions referred to above according to the invention method, a number of factors will generally be considered by the medical practitioner or veterinarian in view of the mammal's age, sex, weight and general condition, as well as the type and extent of the disorder or condition being treated, and the use of other medications, if any, by the mammal receiving the treatment. As such, the administered dose may fall within the ranges or concentrations recited above, or may vary outside, i.e., either below or above, those ranges depending upon the requirements of the individual subject, the severity of the condition being treated, and the particular therapeutic formulation being employed. Determination of a proper dose for a particular situation is within the skill of the medical or veterinary arts. Generally, treatment may be initiated using smaller dosages of the active compound or compounds that are less than optimum for a particular subject. Thereafter, the dosage can be increased by small increments until the optimum effect under the circumstance is reached. For convenience, the total daily dosage may be divided and administered in portions during the day, if desired.

The compounds of formula I and their pharmaceutically acceptable salts can be administered to mammals via either the oral, parenteral (such as subcutaneous, intravenous, intramuscular, intrasternal and infusion techniques), rectal, buccal, topical or intranasal routes. Preferred routes of administration are oral and parenteral. Preferably, administration is in unit dosage form. A unit dosage form of a compound of formula I, or a pharmaceutically acceptable salt thereof, to be used in the methods of this invention may also comprise other compounds useful in the therapy of the disorder or condition for which the compound of formula I or pharmaceutically acceptable salt thereof is being administered or a disorder or condition that is secondary to the disorder condition for which the compound of formula I or pharmaceutically acceptable salt thereof is being administered.

Pharmaceutical compositions containing a compound of the formula I, or a pharmaceutically acceptable salt thereof, are produced by formulating the active compound in unit dosage form with a pharmaceutical carrier. Some examples of unit dosage forms are tablets, capsules, pills, powders, cachets, lozenges, creams, aqueous and nonaqueous oral solutions and suspensions, and parenteral solutions packaged in containers containing either one or some larger number of dosage units and capable of being subdivided into individual doses.

Some examples of suitable pharmaceutical carriers, including pharmaceutical diluents, are gelatin capsules; sugars such as lactose and sucrose; starches such as corn starch and potato starch; cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations.

The compositions to be employed in the methods of this invention can also contain other components such as coloring agents, flavoring agents, and/or preservatives. These materials, if present, are usually used in relatively small amounts. The compositions can, if desired, also contain other therapeutic agents commonly employed to treat the disorder or condition being treated.

The percentage of the active ingredients in the foregoing compositions can be varied within wide limits, but for practical purposes it is preferably present in a concentration of at least 10% in a solid composition and at least 2% in a primary liquid composition. The most satisfactory compositions are those in which a much higher proportion of the active ingredient is present, for example, up to about 95%.

In tablets, the active component is mixed with the carrier having the necessary binding properties in suitable proportions and compacted in the shape and size desired. Powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier, providing a capsule in which the active component, with or without other carriers, is surrounded by a carrier, which is thus in association with it.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection liquid preparations can be formulated in solution in aqueous polyethylene glycol solution. Aqueous solutions suitable for oral use can be prepared by dissolving the active component in water and adding suitable colorants, flavors, stabilizing and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The degree of binding of compounds of the formula I and their pharmaceutically acceptable salts to an α2δ subunit of a calcium channel can be determined using the radioligand binding assay using [³H]gabapentin and the α2δ subunit derived from porcine brain tissue, as described by N. S. Gee et al., J. Biol. Chem., 1996, 271:5879-5776.

The efficacy of pregabalin in treating fibromyalgia was demonstrated in the following in vivo experiment, which is summarized below.

SUMMARY:

Sprague-Dawley male rats given an intramuscular (IM) injection of 100 μL sterile pyrogen-free saline, pH 4 in the gastrocnemius muscle on Days 1 and 5, develop chronic mechanical allodynia (static allodynia) approximately 1 week after the second injection. (Sluka K A, Kalra A, Moore S A. Intramuscular injections of acidic saline produce a bilateral long-lasting hyperalgesia. Muscle & Nerve 2001; 24:37-46.) Allodynia was measured by applying von Frey filaments of varying bending forces (0.41 to 29 g) to the plantar surface of the injected hind limb to determine paw withdrawal threshold (PWT). Evaluations were performed blinded, with the animals and treatments randomized. PWL, 1 to 2 weeks after the second acid saline injection was usually less than 5 g (reduced from 22 to 28 g prior to acid saline injection), representing tactile allodynia. Allodynia was sustained for 3 weeks. Rats injected with pH 7.2 saline showed no allodynia. After acid saline injection, there was no evidence for dynamic allodynia (measured by paw withdrawal to cotton swab stroking) or weight-bearing preference between the hind limbs. Treatment with pregabalin, 10 or 30 mg/kg by oral gavage (PO) 2 weeks after the last acid saline injection, reversed mechanical allodynia. Analgesic-like action of pregabalin was significantly greater than vehicle treatment at 2 to 3 hours after therapy. Morphine, 3 and 10 mg/kg subcutaneously (SC) reduced allodynia, while amitryptiline, 6 mg/kg SC did not alter allodynia. Fibromyalgia is classified by the Americal College of Rheumatology as chronic generalized musculoskeletal pain with allodynia to pressure over a majority of specified tender points. The results with acid saline-injected rats indicate that pregabalin reduces allodynia in a rat model with some similarities to the human disease of fibromyalgia.

Methods:

Acidic Saline-Induced Allodynia: Male Sprague-Dawley rats (Harlan) approximately 350 g were housed in plastic isolators with organic cellulose bedding. Food and water were available ad libidum and animals were maintained on a 12 hr/12 hr light/dark cycle, with testing during the light phase. On test days, rats were placed in a metal chamber on an elevated metal mesh floor and allowed to acclimate for at least 20 minutes. On Day 1, a baseline von Frey filament withdrawal threshold with the right hind paw was obtained. Later on Day 1, acidic saline, pH 4 (100 μL) was injected in the right gastrocnemius muscle and a similar injection was made again in the same location on Day 5. On subsequent days, pain responses (von Frey filament withdrawal threshold, withdrawal in response to light stroking with a cotton swab, and changes in weight bearing between the 2 hind paws) were determined for both hind paws. Experimental drug treatments were evaluated for inhibitory effects on static allodynia and other pain endpoints. Rats with a paw withdrawal threshold (PWT) of 6 g or less on the day of drug testing (Days 14 to 18) were used. Rats were evaluated for PWTs at 1, 2, and 3 hours after receiving either drug or vehicle treatments.

Measurement of Pain-Related Behavioral Responses

Static Allodynia: PWT was determined using von Frey filaments with varying bending forces (0.41, 0.69, 1.2, 2.0, 3.6, 5.5, 8.5, 15.1, and 28.8 g, Stoelting Corp, Wood Dale, Ill.). Pressure was applied to the plantar surface of a hind paw with a single slow application for up to 6 seconds to the plantar surface beginning with the 2.0-g filament. If no withdrawal was obtained, the next higher bending force filament was applied or, in the case of a withdrawal, the next lower force filament was applied. This continued until at least 6 responses were obtained, including at least 1 withdrawal. Withdrawal threshold at each time point (for each rat) then was determined using the Dixon, ‘Up-Down’ method. (Dixon W J. Efficient analysis of experimental observations. Ann Rev Pharmacol Toxicol 1980; 20:441-62.) If no withdrawal was obtained with the 28.8 g filament, a withdrawal threshold of 29 g was assigned.

Dynamic Allodynia: The plantar surfaces of injected and contralateral hind paws were gently stroked with a cotton swab, applied from underneath the wire mesh for up to 15 seconds. Withdrawal time (mean of triplicate values) is reported, with a maximum value of 15 seconds recorded if no withdrawal was observed.

Spontaneous Pain: The rat was placed in a compact clear acrylic plastic box with an elevated platform for the forepaws and a square cutaway in the base for the hind paws. The box was designed to allow contact of the hind paws to each of 2 force transducer plates of an incapacitance tester (Linton Instruments, Norfolk, England), that measured the force applied by each of the hind paws to the floor of the chamber. The weight (in grams) applied to each paw was averaged by the device over a 4-second period and recorded. Reported values are the mean of triplicate readings of the difference in weight applied to the 2 hind paws (contralateral minus injected hind paw).

Results

Characterization of the Model: Two repeated intramuscular injections of acidic saline caused a sustained decrease in the von Frey withdrawal threshold to the planter surface of the previously injected hind limb. These results were similar to those published previously. (Sluka K A, Kalra A, Moore S A. Intramuscular injections of acidic saline produce a bilateral long-lasting hyperalgesia. Muscle & Nerve 2001; 24:37-46.) However, in contrast to the previously published findings, little or no change in withdrawal threshold was observed in the hind limb contralateral to acidic saline injection except at the latest time point tested (Table 1). No changes were observed in withdrawal in response to the cotton swab stimulus or weight bearing endpoints in either hind paw. A representative experiment is shown in Table 2.

Pharmacological Evaluations of Pregabalin in the Acidic Saline Induced Allodynia Model: Rats injected with acidic saline on Days 1 and 5 were evaluated for changes in pain responses on alternate days, beginning at Day 14, following the last acidic saline injection. On a given day, only rats showing allodynia (withdrawal in response to von Frey filaments of 6 g or less) and naïve to previous drug treatments were used to evaluate test compounds. Pregabalin or vehicle (water) was given PO, 30 minutes after baseline paw withdrawal readings. Rats were evaluated at 1, 2, and 3 hours after drug or vehicle treatment. Pregabalin at either 10 or 30 mg/kg PO inhibited static allodynia (measured by von Frey filaments) when tested either 2 or 3 hours after drug treatment (Table 3). Pregabalin treatment at 3 mg/kg PO was without effect on allodynia.

Morphine, 10 mg/kg SC, given 30 minutes after baseline measurements, inhibited static allodynia at 1 and 2 hours after treatment (Table 4). Similar treatment with morphine, 3 mg/kg, increased PWTs, but only at 1 hour (not 2 or 3 hours) after treatment. Amitriptyline, 6 mg/kg SC, did not alter PWTs at 1, 2, or 3 hours posttreatment (Table 5).

Prior repeated injection of pH 4 saline in the gastrocnemius induced mechanical allodynia (measured with von Frey filaments) of several weeks duration to the ipsilateral plantar surface of the hind paw. The same rats did not have dynamic allodynia of the hind paw (in response to cotton swab stroking) or spontaneous pain behavior (a weight bearing preference between the hind paws). Pregabalin 10 and 30 mg/kg PO reduced static allodynia produced by prior acidic saline injections. Morphine 3 and 10 mg/kg SC, reduced static allodynia from prior acidic saline injections. Amitriptyline, 6 mg/kg PO was without inhibitory effects on allodynia. These results agree with previous published results for morphine. (Sluka, K A, Rohlwing J J, Bussey R A, Eikenberry S A, Wilken J M. J Pharmacol. Exp. Ther. 2002, 302:1146-50). Although amitriptyline was without effect in this study, it is often prescribed for fibromyalgia pain, and clinical studies has shown it to be effective. 5,6 It is possible that amitriptyline would be effective in this animal model (with allodynia from repeated acidic saline injections) if it were tested after repeated dosing for several days. This possibility remains to be tested. Higer dosages of amitriptyline were not studied because tachycardia was observed at the 6 mg/kg PO dose and a 10 mg/kg PO dose was lethal in a fraction of rats that were injected.

Static allodynia in the rat hind paw produced by prior repeated injections of acidic saline into the gastrocnemius muscle may provide a method to evaluate novel agents for treating chronic musculoskeletal pain. This animal model may be of use to evaluate experimental analgesic compounds for the treatment of chronic allodynia in syndromes such as fibromyalgia.

TABLE 1
Rat Paw Withdrawal Threshold (PWT) of
the Plantar Hind Paw Surface of the Left
(Ipsilateral) and Right (Contralateral) Side,
Before and After 2 Injections of 100 μL of
Acidic Saline, pH 4.2 on the Left Gastrocnemius Muscle

| | | Day 5 | Day 12 | Day 16 | Day 26 |
|---|---|---|---|---|---|
| | Day 1 | Post- | Post- | Post- | Post- |
| | Before | Initial | Initial | Initial | Initial |
| Hind Limb | Injection | Injection | Injection | Injection | Injection |
| Ipsilateral | 27.47 | 28.84 | 13.19^{a,b} | 7.86^{a,b} | 10.93^{b} |
| PWT | 1.11 | 0.00 | 3.64 | 2.07 | 2.80 |
| SEM | | | | | |
| Contralateral | 28.84 | 25.64 | 28.84 | 28.84 | 17.58^{b} |
| PWT | 0.00 | 2.48 | 0.00 | 0.00 | 3.61 |
| SEM | | | | | |

N = 9, Data are mean values in grams.
^{a}p < 0.05, injected versus contralateral hindlimb, one-way ANOVA on ranks with Tukey test.
^{b}p < 0.05 versus baseline on Day 1, before first injection, one-way ANOVA on ranks with Tukey test.

TABLE 2
Paw Withdrawal Threshold (Ipsilateral), Paw Withdrawal Latency
(Ipsilateral), and Weight Bearing Measurements at Different
Times Before (Day 1) and After (Days 5 and 8) 2 Acid Saline
Injections to the Gastrocnemius Muscle

| | | |
|---|---|---|
| Day 1 | Day 5 Postinitial | Day 18 Post |
| Before Injection | Injection | Initial Injection |

Paw Withdrawal Threshold to von Frey Filaments (g)

| | | | |
|---|---|---|---|
| pH 7.4 | 25.59 | 20.15 | 23.50 |
| SEM | 2.28 | 3.65 | 3.94 |
| pH 4.2 | 27.47 | 12.42a | 9.89a |
| SEM | 1.11 | 3.175 | 2.48 |

Paw Withdrawal Latency to Cotton Swab Stroking (sec)

| | | | |
|---|---|---|---|
| pH 7.4 | 9.39 | 10.83 | 9.11 |
| SEM | 0.65 | 0.34 | 0.93 |
| pH 4.2 | 9.17 | 6.33 | 9.78 |
| SEM | 0.40 | 1.34 | 1.96 |

Weight Bearing: Contralateral Force Minus Ipsilateral Force (g)

| | | | |
|---|---|---|---|
| pH 7.4 | −3.0 | −2.0 | −10.0 |
| SEM | 5.0 | 6.0 | 4.0 |
| pH 4.2 | 7.0 | 5.0 | −13.0 |
| SEM | 5.0 | 5.0 | 14.0 |

ap < 0.05 verus Day 1 by one-way ANOVA on ranks and Tukey test,
n = 6/group.
Data are means values.

TABLE 3
Rat Paw Withdrawal Threshold Before and
After Treatment With Pregabalin PO Following
Prior Repeated Acid Saline Injections to the
Gastrocnemius Muscle^{a}

| | Day 1 | Baseline | 1 hr Post Rx | 2 hr Post Rx | 3 hr Post Rx |
|---|---|---|---|---|---|
| Vehicle | 27.75 | 3.52 | 9.87 | 7.07 | 12.13 |
| SE | 1.09 | 0.35 | 4.05 | 2.24 | 5.04 |
| Pregabalin, 3 mg/kg PO | 27.37 | 5.32 | 7.92 | 4.53 | 6.07 |
| SEM | 1.08 | 0.51 | 2.99 | 1.16 | 1.13 |
| N = 6/group | | | | | |
| Vehicle | 18.65 | 4.72 | 11.70 | 4.17 | 3.74 |
| SEM | 2.50 | 0.90 | 5.78 | 0.70 | 1.42 |
| Pregabalin, 10 mg/kg PO | 24.90 | 3.55 | 2.28 | 28.84^{b} | 22.23^{b} |
| SEM | 3.94 | 0.14 | 0.68 | 0.00 | 6.62 |
| N = 4/group | | | | | |
| Vehicle | 28.84 | 4.57 | 12.95 | 3.61 | 5.05 |
| SEM | 0.00 | 0.73 | 4.47 | 0.62 | 2.19 |
| Pregabalin, 30 mg/kg PO | 26.07 | 4.74 | 18.15 | 25.11^{b} | 23.74^{b} |
| SEM | 1.81 | 0.75 | 4.20 | 2.65 | 4.67 |
| N = 6/group | | | | | |

^{a}Paw withdrawal threshold measured by von Frey filaments, all measurements in grams; all drug treatments given 30 minutes after baseline measurements.
^{b}Significantly different from vehicle group (p < 0.05, 1-way ANOVA on ranks then Tukey Test, all pairwise comparison procedures). Data are mean values.

TABLE 4
Rat Static Allodynia Before and After Treatment With Morphine SC a

| | Day 1 | Baseline | 1 hr Post | 2 hr Post Rx | 3 hr Post Rx |
|---|---|---|---|---|---|
| Vehicle | 28.24 | 4.11 | 4.07 | 9.99 | 9.74 |
| SEM (n = 11) | 0.60 | 0.39 | 0.65 | 3.32 | 2.70 |
| Morphine, 3 mg/kg SC | 24.27 | 2.89 | 20.52^{b} | 9.93 | 7.09 |
| SEM (n = 10) | 1.54 | 0.41 | 3.33 | 3.23 | 2.25 |
| Vehicle | 28.84 | 3.33 | 4.56 | 5.47 | 4.67 |
| SEM (n = 6) | 0.00 | 0.41 | 0.91 | 1.98 | 0.75 |
| Morphine, 10 mg/kg SC | 26.63 | 3.09 | 28.840^{b} | 22.05^{b} | 16.91 |
| SEM (n = 6) | 2.21 | 0.60 | 0.00 | 4.32 | 4.58 |

^{a}Paw withdrawal threshold measured by von Frey filaments, all measurements in grams; all drug treatments given 30 minutes after baseline measurements.
^{b}p < 0.05 versus baseline measurement, 1-way ANOVA on ranks, and Tukey test. Data are mean values.

TABLE 5
Rat Static Allodynia Before and After Treatment With Amitriptyline, SC a

| | Day 1 | Baseline | 1 hr Post Rx | 2 hr Post Rx | 3 hr Post Rx |
|---|---|---|---|---|---|
| Vehicle | 28.84 | 3.06 | 8.64 | 8.64 | 11.50 |
| SEM | 0.00 | 0.28 | 4.27 | 2.02 | 5.52 |
| Amitriptyline, 6 mg/kg | 24.44 | 2.36 | 12.01 | 7.38 | 7.20 |
| SEM | 2.21 | 0.40 | 4.55 | 2.74 | 4.03 |

N = 6/group. There was no significant difference between groups (1 way ANOVA on ranks and Tukey test).
^{a}Drug is given 30 minutes after the baseline measurement.

A clinical study of the effect of pregabalin on human patients with fibromyalgia was also conducted. This study was conducted to assess the efficacy of pregabalin (150, 300, and 450 mg/day) compared with placebo for the relief of pain and improvement in functional status in patients with fibromyalgia. Patients who participated in the study must have met the American College of Rheumatology criteria for fibromyalgia (widespread pain present for at least 3 months, and pain in at least 11 of 18 tender point sites).

Methodology

Following a 1-week baseline phase, qualified patients were randomized to receive either 150, 300, or 450 mg/day pregabalin or placebo according to an 8-week, double-blind, multicenter study design. The intent-to-treat (ITT) population comprised a total of 529 patients: 132 patients received 450 mg/day, 134 received 300 mg/day, 132 received 150 mg/day pregabalin, and 131 received placebo. The first phase of the 8-week double-blind phase consisted of a 1-week titration phase. Patients randomized to the placebo, 150 and 300 mg/day pregabalin treatment groups started out at their fixed dose at Day 1. Patients randomized to 450 mg/day pregabalin treatment group started at 300 mg/day and titrated to the target dose of 450 mg/day on Day 4, and remained at the fixed dose for the remainder of the double-blind period. Following Week 8 of the double-blind phase, patients had the option of entering an open-label follow-on study (Protocol 1008-033).

Criteria for Evaluation

The primary efficacy measurements were derived from the daily, self-assessed pain score from the patient diary. Secondary measures were derived from the SF-MPQ, Manual Tender Point Survey, quality of sleep score from the daily diary, Multidimensional Assessment of Fatigue (MAF), Clinical Global Impression of Change (CGIC) and Patient Global Impression of Change (PGIC), the SF-36 Health Survey (SF-36), Hospital Anxiety and Depression Scale (HADS), and Medical Outcomes Study (MOS) Sleep Scale.

Results

All analyses were performed on the ITT population, defined as all randomized patients who received at least one dose of study medication. The primary efficacy measure, endpoint mean pain score, was significantly better for 450 mg/day pregabalin compared to placebo. A significant difference from placebo was seen in mean pain scores at Week 1 for the 450 mg/day pregabalin group and continued through Week 7. Similar results were seen for the 450 mg/day pregabalin group in most other secondary parameters including: Mean quality of sleep at each week and at endpoint, SF-MPQ sensory, affective, and total scores at endpoint and VAS at endpoint, CGIC, PGIC, and the MAF Global Fatigue Index. A significant difference favoring 450 mg/day pregabalin compared to placebo was seen in the Social Functioning, Bodily Pain, Vitality, and General Health Perception domains of the SF-36 Health Survey. Responder status (defined as the number of patients reporting at least 50% reduction in pain at endpoint compared to baseline) was significantly better for patients in the 450 mg/day pregabalin group compared to placebo (28.9% and 13.2%, respectively; p=0.003). Patients in the 300 and 150 mg/day pregabalin groups were not significantly different from placebo for the primary efficacy parameter. Both 300 and 150 mg/day pregabalin showed significant differences in many of the secondary parameters compared to placebo.

### CONCLUSIONS

Pregabalin was found to be effective at a dose of 450 mg/day in reducing pain associated with fibromyalgia. There was no significant effect on pain at the 150- and 300-mg/day doses. Both the 300 and 450 mg/day pregabalin treatment arms were superior to placebo on improvement in fatigue, clinician and patient global assessments of change, and improvement of sleep quality.

What is claimed is:

1. A method of treating fibromyalgia in a mammal comprising administering to a mammal in need of such treatment a therapeutically effective amount of a compound of the formula I 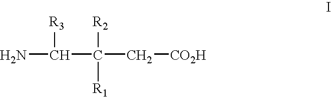
or a pharmaceutically acceptable salt thereof, wherein:
R₁ is a straight or branched unsubstituted alkyl of from 1 to 5 carbon atoms, unsubstituted phenyl, or unsubstituted cycloalkyl of from 3 to 6 carbon atoms;
R₂ is hydrogen or methyl; and
R₃ is hydrogen, methyl, or carboxyl.

2. A method of treating a disorder or condition selected from the group consisting of sleep disorders such as insomnia (e.g., primary insomnia including psychophysiological and idiopathic insomnia, secondary insomnia including insomnia secondary to restless legs syndrome, Parkinson's disease or another chronic disorder, and transient insomnia), somnambulism, sleep deprivation, REM sleep disorders, sleep apnea, hypersomnia, parasomnias, sleep-wake cycle disorders, jet lag, narcolepsy, sleep disorders associated with shift work or irregular work schedules, deficient sleep quality due to a decrease in slow wave sleep caused by medications or other sources, and other sleep disorders in a mammal, comprising administering to a mammal in need of such treatment a therapeutically effective amount of a compound of the formula I 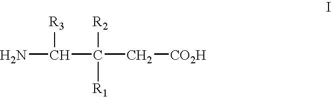
or a pharmaceutically acceptable salt thereof, wherein:
R₁ is a straight or branched unsubstituted alkyl of from 1 to 6 carbon atoms, unsubstituted phenyl, or unsubstituted cycloalkyl of from 3 to 6 carbon atoms;
R₂ is hydrogen or methyl; and
R₃ is hydrogen, methyl, or carboxyl.

3. A method of increasing slow wave sleep in a human subject comprising administering to a human subject in need of such treatment an amount of a compound of the formula I 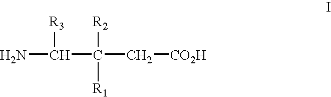
or a pharmaceutically acceptable salt thereof, wherein:
R₁ is a straight or branched unsubstituted alkyl of from 1 to 5 carbon atoms, unsubstituted phenyl, or unsubstituted cycloalkyl of from 3 to 6 carbon atoms;
R₂ is hydrogen or methyl; and
R₃ is hydrogen, methyl, or carboxyl;
That is effective in increasing slow wave sleep.

4. A method of increasing secretion of a human growth hormone in a human subject comprising administering to a human subject in need of such treatment an amount of a compound of the formula I 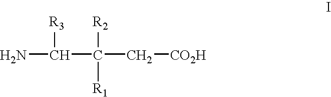
or a pharmaceutically acceptable salt thereof, wherein:
R₁ is a straight or branched unsubstituted alkyl of from 1 to 5 carbon atoms, unsubstituted phenyl, or unsubstituted cycloalkyl of from 3 to 6 carbon atoms;
R₂ is hydrogen or methyl; and
R₃ is hydrogen, methyl, or carboxyl;
that is effective in increasing secretion of a human growth hormone.

5. A method of treating irritable bowel syndrome in a mammal, preferably a human, comprising administering to a human subject in need of such treatment a therapeutically effective amount of a compound of the formula I 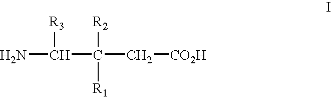
or a pharmaceutically acceptable salt thereof, wherein:
R₁ is a straight or branched unsubstituted alkyl of from 1 to 5 carbon atoms, unsubstituted phenyl, or unsubstituted cycloalkyl of from 3 to 6 carbon atoms;
R₂ is hydrogen or methyl; and
R₃ is hydrogen, methyl, or carboxyl.

6. A method of treating a disorder or condition selected from the group consisting of panic disorder with or without agoraphobia, agoraphobia without history of panic disorder, specific phobias, social anxiety disorder, social phobia, obsessive-compulsive disorder, and stress disorders including post-traumatic stress disorder and acute stress disorder in a mammal, comprising administering to a mammal in need of such treatment a therapeutically effective amount of a compound of the formula I 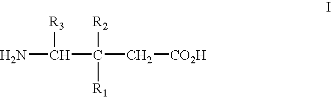
or a pharmaceutically acceptable salt thereof, wherein:
R₁ is a straight or branched unsubstituted alkyl of from 1 to 5 carbon atoms, unsubstituted phenyl, or unsubstituted cycloalkyl of from 3 to 6 carbon atoms;
R₂ is hydrogen or methyl; and
R₃ is hydrogen, methyl, or carboxyl.

7. A method of treating a disorder or condition selected from the group consisting of panic disorder with or without agoraphobia, agoraphobia without history of panic disorder, specific phobias, social anxiety disorder, social phobia, obsessive-compulsive disorder, and stress disorders including post-traumatic stress disorder and acute stress disorder in a mammal, comprising administering to a mammal in need of such treatment:
(a) a compound of the formula I 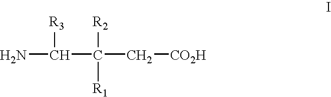
or a pharmaceutically acceptable salt thereof, wherein:
R₁ is a straight or branched unsubstituted alkyl of from 1 to 5 carbon atoms, unsubstituted phenyl, or unsubstituted cycloalkyl of from 3 to 6 carbon atoms;
R₂ is hydrogen or methyl; and
R₃ is hydrogen, methyl, or carboxyl. compound of the formula I, or a pharmaceutically acceptable salt thereof; and
(b) another compound that is an antidepressant or an antianxiety agent, or a pharmaceutically acceptable salt thereof;
wherein the amounts of the active agents “a” and “b” are chosen so as to render the combination therapeutically effective.

8. A method according to claim 7, wherein the disorder or condition being treated is post traumatic stress disorder.

9. A method according to claim 7, wherein the disorder or condition being treated is social phobia or social anxiety disorder.

10. A method of treating two or more disorders or conditions, each of which is independently selected from the group consisting of irritable bowel syndrome, fibromyalgia, neuropathic pain, sleep disorders such as insomnia (e.g., primary insomnia including psychophysiological and idiopathic insomnia, secondary insomnia including insomnia secondary to restless legs syndrome, Parkinson's disease or another chronic disorder, and transient insomnia), somnambulism, sleep deprivation, REM sleep disorders, sleep apnea, hypersomnia, parasomnias, sleep-wake cycle disorders, jet lag, narcolepsy, sleep disorders associated with shift work or irregular work schedules, deficient sleep quality due to a decrease in slow wave sleep caused by medications or other sources, other sleep disorders, panic disorder with or without agoraphobia, agoraphobia without history of panic disorder, specific phobias, social anxiety disorder, social phobia, obsessive-compulsive disorder, and stress disorders including post-traumatic stress disorder and acute stress disorder in a mammal, comprising administering to a mammal in need of such treatment a therapeutically effective amount of a compound of the formula I 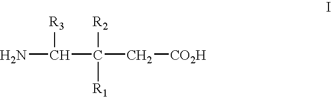
or a pharmaceutically acceptable salt thereof, wherein:
R₁ is a straight or branched unsubstituted alkyl of from 1 to 5 carbon atoms, unsubstituted phenyl, or unsubstituted cycloalkyl of from 3 to 6 carbon atoms;
R₂ is hydrogen or methyl; and
R₃ is hydrogen, methyl, or carboxyl.

11. A method according to claim 1 wherein a compound of the formula I, or a pharmaceutically acceptable salt thereof, is administered to a human for the treatment of fibromyalgia and a concomitant disorder or condition selected from panic disorder, irritable bowel syndrome, functional abdominal pain, neuropathic pain, major depressive disorder and dysthymia.

12. A method according to claim 1 wherein a compound of the formula I, or a pharmaceutically acceptable salt thereof, is administered to a human for the treatment of fibromyalgia and a concomitant somatoform disorder selected from somatization disorder, conversion disorder, body dysmorphic disorder, hypochondriasis, somatoform pain disorder, undifferentiated somatoform disorder and somatoform disorder not otherwise specified.

13. A method of increasing slow wave sleep in a human subject being treated with an active pharmaceutical agent that decreases slow wave sleep comprising administering to such human subject a therapeutically effective amount of a compound of the formula I or a pharmaceutically acceptable salt thereof.

14. A method according to claim 14 wherein the active pharmaceutical agent that decreases slow wave sleep is morphine.

15. A method of increasing slow wave sleep in a human subject comprising administering to a human subject in need of such treatment:
(a) a compound of the formula I or a pharmaceutically acceptable salt thereof; and
(b) a human growth hormone or human growth hormone secretagogue, or a pharmaceutically acceptable salt thereof;
wherein the amounts of the active agents “a” and “b” are chosen so as to render the combination effective in increasing slow wave sleep.

16. A method of increasing slow wave sleep in a human subject being treated with an active pharmaceutical agent that decreases slow wave sleep comprising administering to a human subject in need of such treatment:
(a) a compound of the formula I or a pharmaceutically acceptable salt thereof; and
(b) a human growth hormone or human growth hormone secretagogue, or a pharmaceutically acceptable salt thereof;
wherein the amounts of the active agents “a” and “b” are chosen so as to render the combination effective in increasing slow wave sleep.

17. A method according to claim 1 wherein the compound of formula I that is administered is pregabalin.

18. A method according to claim 2 wherein the compound of formula I that is administered is pregabalin.

19. A method according to claim 3 wherein the compound of formula I that is administered is pregabalin.

20. A method according to claim 4 wherein the compound of formula I that is administered is pregabalin.

21. A method according to claim 5 wherein the compound of formula I that is administered is pregabalin.

22. A method according to claim 6 wherein the compound of formula I that is administered is pregabalin.

23. A method according to claim 7 wherein the compound of formula I that is administered is pregabalin.

24. A method according to claim 8 wherein the compound of formula I that is administered is pregabalin.

25. A method according to claim 11 wherein the compound of formula I that is administered is pregabalin.

26. A method according to claim 16 wherein the compound of formula I that is administered is pregabalin.

27. A method according to claim 17 wherein the compound of formula I that is administered is pregabalin.
